(19) United States
(12) Patent Application Publication
(10) Pub. No.: US 2004/0236083 A1
(43) Pub. Date: Nov. 25, 2004

(54) Protein separation column

(76) Inventors: Sharon Ann Libert, Hockessin, DE (US); Victor Sisto Lusvardi, Chadds Ford, PA (US); Timothy Tucker Norton, Avondale, PA (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/835,514

(22) Filed: Apr. 29, 2004

Related U.S. Application Data
(60) Provisional application No. 60/470,307, filed on May 14, 2003.

(51) Int. Cl. C07K 1/12

(52) U.S. Cl. 530/412; 210/656

(57) ABSTRACT

The present invention relates to columns for carrying out separation processes involving protein solutions, wherein at least the interior surface of the column is comprised of fluoropolymer.

### BACKGROUND OF THE INVENTION

1. Field of the Invention

This invention relates to the separation of dissolved components such as proteins from protein solutions without contaminating the protein.

2. Description of Related Art

In the biotechnology industry in which proteins are therapeutic agents, i.e. drugs, these proteins are obtained by bioprocessing operations, the proteins are obtained as very dilute solutions, e.g. often no more than 1 g/liter of the therapeutic protein, in an aqueous medium, sometimes as little as 1 ppm of the therapeutic protein. Often other proteins, having been co-produced with the therapeutic protein, are also present in the solution as dissolved components. The solution is then treated to separate the therapeutic protein from the solvent of the solution (or simply from the solution) and from any undesired protein that may be present in the solution. The separation is typically carried out by contacting an adsorptive matrix material, which adsorbs either the therapeutic protein or an undesired protein, if present, either to separate the therapeutic protein from the solution, thereby obtaining it in concentrated form, or to remove undesired protein from the solution to thereby purify the therapeutic protein remaining in the solution, respectively.

Typically, the matrix material comprises a binding protein such as protein A or protein G, being present on a substrate. The binding protein binds the target protein, or other target dissolved component present in the solution to the substrate, and the substrate permits the protein solution to flow through it, with the flow being in such tortuous passages or such fine passages or both, that the target protein or other target dissolved component comes into intimate contact with the binding protein present on the substrate, to thereby be adsorbed on its surface, including pores within the substrate, while the solution passes through the substrate. The combination of binding protein and substrate is typically called an affinity matrix. The substrate of the affinity matrix can be in a variety of different forms, e.g. as packing in a vessel such as a separation column, or porous membrane bridging the interior of the vessel in which the separation is carried out. The packing can be in such forms as particulate material such as beads or gels or monoliths, i.e. an extruded length of porous polymeric material. The substrate can include paramagnetic material for slurrying in the protein solution. Other mechanisms of adsorption of the adsorbent matrix material include size exclusion chromatography, ion exchange chromatography, hydrophobic interaction chromatography, and filtration using a porous membrane. Following adsorption, the target protein or other target dissolved component adsorbed on the matrix material is then eluted from the matrix material, i.e. by contacting the affinity matrix with an eluting liquid that removes the adsorbed material (protein or other dissolved component) from the matrix material.

The vessel in which the treatment of the protein solution is carried out is subjected to one or more corrosive liquid environments, depending on the adsorption method and particular dissolved component to be removed from solution. For example, the adsorption matrix material is contacted with concentrated salt solution at low pH to increase the effectiveness of adsorption. This salt solution is normally added to the protein solution or to the vessel simultaneously with the feed of the protein solution to the vessel. Elution of the adsorbed component, e.g. the target protein, from the adsorption matrix material may also be carried out with a concentrated salt solution of high pH. When concentrated salt solution is not used to promote adsorption, it is often used to cause elution. Periodically, the interior surface of the vessel, which is the surface exposed to these corrosive liquid environments, is cleaned by washing with strong caustic solution, another corrosive liquid environment. It is critical, however, that the interior surface vessel in which the treatment is carried out not be corroded by the liquid environment, so that the solution does not become contaminated, thereby contaminating the therapeutic protein.

The bioprocessing industry has addressed this criticality by using corrosion-resistant metal alloys as the material of construction of the vessels and forming the interior surface thereof. M. Gonzales, “Stainless Steel Tubing in the Biotechnology Industry” Biotechnology/Pharmaceutical facilities design, Apr. 30, 2001 discloses that stainless steels are non-corroding and non-contaminating such that they can withstand heat and sterilization treatments and are easily welded to thereby satisfy the stringent requirements of bioprocessing. While considering stainless steel to be non-corroding and non-contaminating, the biotechnology industry has nevertheless encountered corrosion and addressed this by shifting to using more corrosion resistant metals. Stainless steel 316L, having a maximum of 0.03 wt % C has been the most widely used. If corrosion is detected on the metal alloy polished surface or in the metal alloy welds or in the liquid environment of the separation steps, the stainless steel is changed to AL6XN, low carbon, high purity superaustenitic stainless steel (21 wt % Cr, 24 wt % Ni, 6.5 wt % Mo, 0.21 wt % Ni, no more than 0.03 wt % C, with the remainder to total 100 wt % being Fe) which has greater corrosion resistance than 316L. If the corrosion resistance of AL6XN is insufficient, then Inconel® 625 (I625) is used (Ni 61 wt %, Cr 21.5 wt %, Mo 9 wt %, Fe 2.5 wt %, Cb+Ta 3.7 wt % and small amounts of other elements). If still greater corrosion resistance is needed, then Hastelloy® C276 is used (Ni 57 wt %, Cr 15.5 wt %, Mo 16 wt %, Fe 5.5 wt %, W 3.75 wt %, Co 1.25 wt % max, Mn 0.5 wt % max). The corrosion problem can be exacerbated when the temperature and/or pH of the liquid environment is changed to improve the effectiveness of the separation or cleaning.

Though the corrosivity aspect of the stainless steel has been addressed, by using more corrosion resistant metal alloys, the aspect of contamination of the protein solution, even when the more corrosion-resistant metal alloys have been used, has generally not been examined.

### BRIEF SUMMARY OF THE INVENTION

It has been found that even the best corrosion-resistant metal alloys are deficient for inertness to the liquid environment of the separation process under conditions that can be commonly encountered, i.e. the metal alloy vessels contaminate the protein solution with corrosion products containing metals from which the vessel is made. Metal contamination is intolerable; although metal contamination might be low in absolute terms, its presence can deleteriously affect the therapeutic protein. In the case of the therapeutic protein being adsorbed by and thus concentrated on the adsorptive matrix material, the presence of metal contamination in the liquid environment has adverse affects ranging from loss of yield of the highly valuable protein to spoilage of the therapeutic value of the protein altogether or to render the protein undesirable for consumption. This deficiency has been found to exist for polished surfaces of the various corrosion-resistant metal alloys used. A greater concern is the metal contamination arising from welds used to fabricate the vessel and necessarily exposed to the interior of the vessel, i.e. forming a portion of the interior surface. The welds are more susceptible to corrosion attack by the liquid environment than the polished surfaces. This is so because the composition and microstructure of the weld may be different than that of the base metal. In addition, the weld may be more susceptible to cracking as a result of residual stresses, physical defects, and altered mechanical properties. The combination of altered mechanical properties and compositional differences can result in a greater susceptibility to environmental-induced cracking.

The present invention has found that superior corrosion resistance to the liquid environment arises when the interior surface of the vessel in which the protein solution is treated comprises fluoropolymer. The fluoropolymer does not attract the protein or the solution containing it and provides little to no contamination of the liquid environment with metals such as Cr, Ni, Fe, Mn or Mo, the most common metals, notwithstanding the fact the fluoropolymers are made in metal reactors and melt processed to form pellets in metal equipment at temperatures exceeding 350° C., such that the fluoropolymer itself may contain metal contaminants. U.S. Pat. No. 6,541,588 B1 discloses fluoropolymer melt pellets containing over 300 ppm of the sum of Fe, Ni, and Cr metals (Table 1). Melt pellets are the usual starting material for melt-fabrication of articles for various utilities and for the interior surface of the vessel used in the present invention.

Thus, the vessel of the present invention can be described as apparatus for separating a dissolved component from a protein solution, comprising a column having an interior surface, said interior surface comprising fluoropolymer, and adsorbent matrix material positioned in said column for separating said dissolved component from said solution.

The test for determining metal contamination is disclosed in the Examples. It has been found that the metal contamination levels arising from the various corrosion-resistance alloy materials described previously ranges from about 1900 to over 10,000 ng/cm². These contamination metals are not detected in the liquid environment used in the test when the apparatus of the present invention is used, the limits of detection being 32 ng/cm² (total of all of the above-mentioned metal contaminants ). Thus, insofar as metal contamination of the solution arising from the fluoropolymer is concerned, the solution is essentially free of metal contamination.

Besides the advantage of the fluoropolymer vessel interior surface not contaminating the liquid separation environment, the fluoropolymer material of construction has the additional advantage that it can be welded together without the use of welding material, i.e. material of different composition. The fluoropolymer sticks to itself and welds together when heated sufficiently and pressure is applied to force together the surfaces of the fluoropolymer to bond to one another. Thus, the weld is as non-contaminating as the surface of the fluoropolymer forming the interior surface of the treatment vessel.

Another advantage of the fluoropolymer vessel interior surface is that proteins are less likely to stick to the fluoropolymer surface than a metal alloy surface since proteins are known to stick to metal surfaces. The protein yields will be greater with vessels constructed with fluoropolymer interior surfaces.

### DETAILED DESCRIPTION OF THE INVENTION

The apparatus of the present invention uses known separation techniques as described above for separating a dissolved component contained in a protein solution, from the solution. Thus, according to one embodiment of the present invention, the separating includes contacting the protein solution with adsorptive matrix material to adsorb said protein from said solution, thereby separating the protein from the solution. The adsorption is obtained by known means as described above, e.g. by the adsorptive matrix material including at least one adsorbent to form an affinity matrix or wherein the matrix material itself provides the adsorbent function either through chemical interaction or by size exclusion. Protein A and Protein G are the most common binding proteins used in the affinity matrix. The adsorbent is selected according to effectiveness for attracting the particular protein desired. “Protein” as used herein is used in the broadest sense to include peptide, polypeptide, long chain polypeptides, e.g. containing at last 20 amino acid units, often simply called proteins, and heteroproteins, such as glycoproteins and phosphoproteins. Usually the target protein will be the therapeutic protein, it being adsorbed on the adsorbent matrix material. The target protein, however, can be an undesirable dissolved component of the protein solution, such as another protein constituting a virus, which can be separated from the therapeutic protein by the adsorption matrix material, leaving the therapeutic protein still in solution, which is thus rendered free of undesirable protein or with a much smaller concentration of the virus remaining. The therapeutic protein can then be separated from the remaining solution by repeating the process of the present invention, using an adsorbent which targets the therapeutic protein.

Known forms of the adsorption matrix material can be used, e.g. beads, gel, monoliths and porous membranes, all achieving intimate contact with the dissolved target protein such as the therapeutic protein as the solution passes through the adsorbent matrix material. By adsorption is meant that the target dissolved component is either attracted to, trapped within, or excluded on or by the surface of the matrix material, whether the surface be an outside surface of the matrix material or internal surfaces such as surfaces of cracks and fissures, and surfaces of pores within the matrix material, and the walls of cells in open-cell monoliths, or simply the pores in a porous membrane. Beads and gel are typically used in chromatographic separations, while porous membrane is used more often in filtration type of separations. Multiple porous membranes can be used. Gel packing in chromatographic separation uses size exclusion as a way to cause the target protein to adsorb on the gel matrix material. Monolith packing is available in the form of an extruded porous plug of plastic material foamed with open-cell structure of having pores being open so that when the monolith is inserted into the vessel, such as in the form of a column, the protein solution flows through the monolith via the open cells or pores, leaving the target protein adsorbed in the pores of the monolith.

After the adsorption step is carried out, the separation can be continued by said eluting of the target dissolved component, e.g. protein, adsorbed on said matrix material to remove it from said matrix material. The separation step can include the use of corrosive liquids creating corrosive liquid environment in the interior of the vessel in which the separation is carried out. The particular liquids used will depend on the separation being carried out. By way of example, the adsorption can be aided by contacting the adsorbent matrix material with concentrated salt solution at low pH. The elution of the target dissolved component held by the matrix material can be obtained by contacting the matrix material with an elution liquid, which may also be a concentrated salt solution but at high pH. Alternatively, the separation may involve the use of high pH salt solution to aid binding, followed by the use of low pH salt solution for elution. The difference between the binding step and the elution step may only involve change in salt concentration. In the embodiment wherein the adsorption is done by a binding protein, i.e. using an affinity matrix, the elution liquid contacts the adsorbed protein and removes it from the matrix material. Periodically, the vessel is then cleaned by washing with concentrated caustic solution. These steps can be carried out at room temperature (20° C.), but higher temperatures are preferred for increased productivity. The adsorbent matrix material may be cleaned along with the interior surface of the vessel and may be discarded as desired to maintain the effectiveness of the separation and the purity of the system. Fresh adsorbent matrix material can then be positioned within the vessel to accomplish further separation processing.

In accordance with the present invention, the interior surface of the vessel within which the separation is carried out comprises fluoropolymer. The entire vessel can be made of fluoropolymer, depending on the size of the vessel, but for commercial size vessels, the vessel will typically be a metal vessel lined with fluoropolymer. Fluoropolymer linings for vessels, columns, pipe, valves, heat exchangers, hoses, etc. are disclosed on pp. 311-326 of Modern Fluoropolymers, High Performance Polymers for Diverse Applications, edited by J. Scheirs, published by John Wiley & Sons (1998). As disclosed therein, various methods are available for obtaining the lining, including but not limited to adhesive bonded, fabric-backed sheets of fluoropolymer, adhesive bonded, etched sheets of fluoropolymer, loose lining of sheets of fluoropolymer welded together, rotolining using fluoropolymer in powder, bead or resin form, spray and baked powder coating of fluoropolymer, and liquid fluoropolymer coating systems. Typically the lining will be about 0.25 to 5.1 mm thick, but preferably at least about 2 mm thick to withstand the loading and unloading of adsorptive matrix material in the form of packing, without puncture of the lining thickness. While fluoropolymer linings are known for corrosion resistance, the unique nature of the protein separation process and the protein itself, including its chemistry, its extremely high value, and its availability only in very dilute solutions, have kept the bioprocessing industry focused on metal vessels, including metal interior surfaces. It is also known that fluoropolymers, because they are made in metal equipment and processed (melt-fabricated) at extremely high temperatures, exceeding 300° C., contain metal contaminant. U.S. Pat. No. 6,541,588 discloses reducing the metal contamination of a fluoropolymer occurring in melt processing from above 300 ppm, by fluorine treating the fluoropolymer prior to melt extrusion to form pellets, but this step is unnecessary in the practice of the present invention.

The vessel in which the separation is carried out can also be called a column, i.e. having a generally cylindrical shape and length ranging from less than the diameter of the cylindrical shape to greater than the diameter. Thus the column can have the appearance of a tank. The cylindrical shape is generally positioned to be vertical, and the cylindrical shape can be circular or different annular shape. The protein solution input and output to the column can be in accordance with batch or continuous operation of the column. In continuous operation, the solution input can be at the top of the column and the output from the bottom, i.e. downflow mode. Alternatively, the operation can be upflow, wherein the solution input is at the bottom of the column, to flow upwardly through the adsorbent matrix material and to exit the column at the top of the column. The methods of continuous chromatography are well known to those skilled in the art and include moving bed and simulated moving bed techniques. The inlet opening and outlet opening are arranged in the top and bottom closures of the column accordingly. One form of batch operated column is a centrifugal tube, wherein the top end has an inlet opening for the protein solution and the bottom of the column is closed to permit the solution to be collected at the bottom of the column. In all cases, adsorptive matrix material is positioned within the column to intimately contact and/or intercept the solution as it traverses the length of the column to carry out the desired adsorptive separation of the target dissolved component from its solution. The matrix material used in the centrifugal tube can be a porous membrane, typically operating by size exclusion, but possibly assisted by including a binding protein or other interactive species on or making up the membrane. The application of centrifugal force causes the solution to pass through the membrane, leaving the target dissolved component on the membrane. The centrifugal tube embodiment of the present invention will typically be entirely of fluoropolymer, while the larger columns used for continuous operation will comprise a shell and a lining of the fluoropolymer to form the interior surface of the column. The shell will provide the required mechanical strength for the column, while the fluoropolymer lining provides the freedom from metal contamination desired. Preferably the shell will be made of metal, but can be made any material that provides the mechanical strength necessary, without the need for expensive corrosion-resistant materials, because the shell is no longer in contact with the protein separation system. In the case of the embodiment of the present invention wherein a shell is present, the lining to form the interior surface can be accomplished as described above.

The fluoropolymer used in the present invention is preferably, but not limited to, melt-flowable fluoropolymer for ease of fabrication and welding together in fabrication processes requiring welding to complete the lining. The fluoropolymers are also preferably partially crystalline, i.e. they have a melting point. The melting point is preferably at least about 225° C., more preferably at least about 250° C., and most preferably 250-315° C. and may be as high as 343° C. One group of preferred fluoropolymers is the perfluorinated polymers, i.e., homopolymers of tetrafluoroethylene (TFE) and copolymers of tetrafluoroethylene (TFE) with perfluorinated monomer. The copolymer can include one or more of such perfluorinated comonomer. Examples of pertluorinated monomers include perfluoroolefins containing 3-8 carbon atoms, such as hexafluoropropylene (HFP), and perfluoro(alkyl vinyl ether) (PAVE), wherein the alkyl group contains 1 to 5 carbon atoms. Examples of such vinyl ethers include perfluoro(methyl, ethyl, and propyl vinyl ether). Copolymers of TFE and PAVE are commonly available as PFA copolymers, including MFA copolymer, which is a copolymer of TFE with perfluoro(methyl vinyl ether) and at least one additional vinyl ether, such as perfluoro(propyl vinyl ether), and such copolymers typically contain 1 to 10 wt % of the PAVE copolymerized monomer. PFA copolymers have a melting point typically in the range of 300 to 310° C. and melt flow rate (MFR) of 1 to 50 g/10 min, both determined in accordance with ASTM D 3307-93. Copolymers of TFE and HFP are commonly available as FEP copolymers. Typically the HFP content of the copolymer will be characterized by an hexafluoropropylene Index (HFPI) of about 2.0-5.3. HFPI is the ratio of two infrared absorbances measured on a film of the copolymer, which can be converted to wt % HFP by multiplying by 3.2 as disclosed in the paragraph bridging cols. 3 and 4 of U.S. Pat. No. 5,703,185. Preferably, the TFE/HFP copolymer contains at least one additional copolymerized monomer such as PAVE in a amount effective for the copolymer to exhibit an MIT flex life to be at least about 2000 cycles, preferably at least about 4000 cycles. Measurement of MIT flex life is disclosed in U.S. Pat. No. 5,703,185. Generally the amount of such additional monomer will be from about 0.2 to 3 wt %, based on the total weight of the copolymer. One preferred PAVE is perfluoro(propyl vinyl ether) and the most preferred PAVE is perfluoro(ethyl vinyl ether). FEP copolymers typically exhibit a melt flow rate (MFR) of 1 to 50 g/l 0 min and melting point of 250 to 280° C., both determined in accordance with ASTM D2116-91a.

Amorphous fluoropolymers may also be used in accordance with this invention. Amorphous polymers do not have crystalline melting points but are characterized by their glass transition temperatures (Tg). Such fluoropolymers for use in the present invention will be non-elastomeric, having a Tg greater than 0° C. The Tg of an amorphous polymer used according to this invention should be at least about the same as the temperatures to which the polymer will be exposed in the course of the process of this invention. Teflon® AF is a commercial amorphous perfluoropolymer (E. I. du Pont de Nemours & Co., Inc., Wilmington Del. USA) available with Tg of 160° C. and of 240° C. An advantage of amorphous fluoropolymers is that they may be applied as coatings from solution. Damage to these coatings in the course of use is easily repaired by further application of fluoropolymer from solution. For purposes of repair, the Tg of the amorphous fluoropolymer need not be as high as the temperatures to which the polymer will be exposed.

Preferably, the perfluoropolymer is also fluorine treated to form —CF₃ end groups, from such end groups as —CF₂CH₂OH, —CONH₂, —COOH, and —COF endgroups to avoid bubbles that may form during melt fabrication of the sheeting for the lining or the lining itself. Preferably, after fluorination, the fluoropolymer has fewer than 50 of such groups (in total) per 10⁶ carbon atoms and more preferably less than 20. Fluorination of crystalline fluoropolymers is disclosed in U.S. Pat. No. 4,723,658. Fluorination of amorphous fluoropolymers is disclosed in U.S. Pat. No. 5,045,605. The fluorine treatment is preferably carried out on pellets of the fluoropolymer that have been formed by melt extrusion and cutting up into the pellet shape. The pellets are then melt-fabricated into the entire vessel or just the lining to form the interior surface of the vessel. In the case of rotolining, however, wherein the only melt fabrication involved is the melting of powder of the fluoropolymer against the rotating shell of the vessel heated above the melting temperature of the fluoropolymer to form the lining, the powder can be fluorine-treated; extrusion molded pellets are never formed. For large columns, however, fluorine-treated pellets are used to first form the lining and then the lining is secured to the interior surface of the shell.

In addition to the perfluorinated thermoplastic tetrafluoroethylene copolymers described above, such fluorinated thermoplastic (melt-fabricable) polymers as ethylene/tetrafluoroethylene copolymers (ETFE) and ethylene/chlorotrifuoroethylene (ECTFE) can also be used in the present invention, with ETFE being preferred. Such ETFE is a copolymer of ethylene and tetrafluoroethylene, preferably containing minor proportions of one or more additional monomers to improve the copolymer properties, such as stress crack resistance. U.S. Pat. No. 3,624,250 discloses such polymers. The molar ratio of E (ethylene) to TFE (tetrafluoroethylene) is from about 40:60 to about 60:40, preferably about 45:55 to about 55:45. The copolymer also preferably contains about 0.1 to about 10 mole % of at least one copolymerizable vinyl monomer that provides a side chain containing at least 2 carbon atoms. Perfluoroalkyl ethylene is such a vinyl monomer, perfluorobutyl ethylene being a preferred monomer. The polymer has a melting point of from about 250° C. to about 270° C., preferably about 255° C. to about 270° C. Melting point is determined according to the procedure of ASTM 3159. Preferably, the ETFE used in the present invention has a melt flow rate (MFR) of 1 to 50 g/10 min as determined in accordance with the ASTM 3159 procedure. Vinylidene fluoride polymer can also be used, provided that precautions are taken because of the greater susceptibility to attraction of dissolved components from the solution and to attack by caustic cleaning solutions in the presence of solvents. The preferred fluoropolymer used in the present invention is perfluoropolymer, i.e. perfluorinated polymer.

### EXAMPLES

Metal alloys and fluoropolymer corrosion evaluations are conducted using two methods shown in Example 1 and Example 2. The methods used are a metal extraction technique developed as part of the present invention for the semi-conductor industry to determine metal contamination levels from process components and the procedure of ASTM G48-00 Pitting and Crevice Corrosion Resistance of Stainless Steels and Related Alloys by Use of Ferric Chloride Solution, respectively. The salt solution used in both methods described in Example 1 and Example 2 is a 15 wt % aqueous solution of sodium chloride (NaCI) that is adjusted to pH 2 by addition of hydrochloric acid to the medium.

### Example 1

In this example the effect of salt solution on the polished surface of metals is determined. Test coupons are provided for all the test materials free of crevices. The size of each metal coupon is 2 inches by 2 inches by 0.125 inches thick (2.54 cm by 2.54 cm by 0.3 cm). The PFA test coupon size is 2 inches by 2 inches by 0.060 inches thick (2.54 cm by 2.54 cm by 0.16 cm). Each test coupon is immersed into the test solution described above for a time period of seven days and maintained at a temperature of 40° C. After the 7 day exposure the metal contamination level in the test solution is measured and reported as ng per cm² of coupon. The metal contamination in the salt solution is determined by high resolution inductively coupled plasma mass spectrometry (ICP-MS). The coupon immersion and metal analysis is performed in a clean room environment by CHEM TRACE® Company, 44050 Fremont Blvd, Fremont, Calif. 94538, USA. The salt solution before suspension of the coupons therein is analyzed to reveal that none of the metals Cr, Ni, Mo, Mn, and Fe are detectable in the solution. The results are shown in Table 1.

TABLE 1
Metal Extraction (ng of metal/cm² of coupon)

| | |
|---|---|
| Metal Alloys | Detection |

| Element | 316L | AL6XN | I625 | C276 | PFA | Limit |
|---|---|---|---|---|---|---|
| Fe | 8550 | 2750 | 405 | 595 | b/d | 10 |
| Ni | 845 | 775 | 1200 | 2600 | b/d | 10 |
| Cr | 950 | 375 | 190 | 260 | b/d | 10 |
| Mo | 94 | 145 | 99 | 615 | b/d | 1 |
| Mn | 205 | 17 | 4 | 22 | b/d | 1 |

The PFA is Teflon® PFA HP fluoropolymer available from E. I. du Pont de Nemours and Company, Wilmington Del. USA. The PFA fluoropolymer is well known as a copolymer of tetrafluoroethylene with sufficient perfluoro(alkyl vinyl ether) to make the fluoropolymer melt flowable. While the metal contamination of the metal coupons diminishes in contaminating the salt solution as the alloy increases in cost, the contamination nevertheless varies with the amount of particular metals being present in the alloy and exceeds 200 ng/cm². In contrast, the metal contamination from the PFA coupon is so low that it is not detectable (b/d=below limits of detection).

### Example 2

In this Example, the effect of temperature on metal corrosion in salt solution is determined in accordance with ASTM G48 method D. The test coupons are suspended in the salt solution for 72 hours at a constant temperature and examined for crevice corrosion. The temperature is increased in 10 degree increments until crevice corrosion is observed. The test coupons are fitted with multiple crevice washers on both sides of the coupon and torqued to 20 in·lb (2.26 N·m). The coupons are tested in independent test tubes containing 500 ml of solution. The corrosion testing starts at 25° C. for 316L and 35 ° C. for all other alloys. The results are shown in Table 2.

TABLE 2
Temperature at which Crevice Corrosion Occurs

| Alloy | Temperature - ° C. |
|---|---|
| 316L | 35 |
| AL6XN | 60 |
| I625 | 60 |
| C276 | 80 |
| PFA | >85 |

The PFA fluoropolymer shows no indication of crevice corrosion at 80° C., so the test is extended to an additional exposure of one week at 85° C. in the salt solution, and still no crevice corrosion is visible. Because this test already indicates the superiority of the PFA fluoropolymer, the test is discontinued.

### Example 3

500 μl of bacterial suspension containing a fusion protein of Protein A—insulin growth factor is injected into a 316L stainless steel chromatography column (2.5 cm length, 0.5 cm diameter), the interior surface of which is coated with Teflon® AF fluoropolymer and is packed with IgG Sepharose 6 Fast Flow (Amersham Pharmacia, Piscataway, N.J.) media. After the sample is injected, approximately ten column volumes of aqueous binding buffer (pH 7.5 composed of 0.05 M Tris-HCl with 0.05% Tween 20) is added to the column, followed by five column volumes of aqueous wash buffer (pH 4.6, consisting of 10 mM ammonium acetate). After rinsing, the adsorbed fusion protein is eluted by five column volumes of aqueous elution buffer (pH 3.2, consisting of 0.2 M acetic acid).

The 316L stainless steel chromatography column with interior surface coated with Teflon® AF is highly resistant to corrosion and to leaching of metal which would deleteriously affect the protein. No metal contamination is detectable in any of the solutions exiting the column.

The fluoropolymer-coated column described above is made by coating the interior surface of the stainless steel tube with a solution of the fluoropolymer (fill and drain), followed by drying and heating to drive off the solvent, thereby forming the fluoropolymer lining of the tube.

### Example 4

The column is prepared from a tank composed of a cylindrical sidewall 1.5 m in diameter and 1 m tall, with the top and bottom being closed by dome-shaped closures, to form the shell of the column made of stainless steel. The top closure is equipped with an inlet port for feeding protein solution into the interior of the column, and the bottom closure is equipped with an outlet port through which the solution exits the column, the dome shape of the bottom closure directing the solution to this port. A porous tray bridges the bottom closure and beads of affinity matrix material are retained on this tray. The beads fill up the interior of the column to just below the inlet port. Positioned between the inlet port and the top surface of the bed of beads is a distribution manifold for distributing the protein solution across the entire top of the bed of beads. The distribution manifold and tray are made of fluoropolymer. The interior surface of the column is lined with the PFA fluoropolymer tested in Examples 1 and 2. The lining of the cylinder portion is by sheet of the fluoropolymer laminated to glass fabric, the latter facing the interior surface of the cylinder and glued to this surface. The sheet is 2 mm thick. The interior surface of the top and bottom closures are lined with thermoformed glass-fabric backed sheet of the same thickness, glued to their respective closures. The inlet and outlet openings are also lined with fluoropolymer. The seams between abutting edges of the fluoropolymer sheet are welded together by the PFA fluoropolymer itself. The junction between the linings of the bottom closure and the cylindrical shell is also butt welded together, while the top closure is removable and is clamped in place with a fluoropolymer seal filling the gap between the lining of the cylindrical shell and the top closure. The lining of the closures are also welded to the linings of the inlet and outlet ports.

What is claimed is:

1. Apparatus for separating a dissolved component from a protein solution, comprising a column having an interior surface, said interior surface comprising fluoropolymer, and adsorbent matrix material positioned in said column for separating said dissolved component from said solution.

2. The apparatus of claim 1 wherein said dissolved component is a virus.

3. The apparatus of claim 1 wherein said dissolved component is a protein.

4. The apparatus of claim 1 wherein said column is entirely of said fluoropolymer.

5. The apparatus of claim 1 wherein said column includes said interior surface as a lining.

6. The apparatus of claim 1 wherein column has a bottom end which is open, whereby said solution can exit said column through said bottom end.

7. The apparatus of claim 1 wherein said column has a bottom end which is closed, whereby said solution collects at the bottom end of said column.

8. The apparatus of claim 1 wherein said adsorbent matrix material comprises an affinity material for binding said dissolved component to said matrix material.

9. The apparatus of claim 1 wherein said adsorbent matrix material comprises an ion exchange matrix material.

10. The apparatus of claim 1 wherein adsorbent matrix material comprises size exclusion matrix material.

11. The apparatus of claim 1 wherein said adsorbent matrix material comprises a porous membrane.
